(19) United States
(12) Patent Application Publication
(10) Pub. No.: US 2017/0128282 A1
(43) Pub. Date: May 11, 2017

(54) TRANSPIRATION LEAK PROTECTION ELEMENT

(71) Applicant: Gerhard Goss, Berikon (CH)

(72) Inventor: Gerhard Goss, Berikon (CH)

(21) Appl. No.: 15/319,408

(22) PCT Filed: May 22, 2015
(86) PCT No.: PCT/IB2015/053768

§ 371 Date: Dec. 16, 2016

(30) Foreign Application Priority Data
Jun. 25, 2014 (CH) 00966/14

(51) Int. Cl. A61F 13/505; A41B 9/12

(52) U.S. Cl. CPC A61F 13/505; A41B 9/12

(57) ABSTRACT

The invention relates to a transpiration leak protection element. which consists of a moisture-proof membrane layer, a cover layer of a smaller size which is attached thereto, and a moisture-permeable pocket filled with a highly absorbent material.

The present invention relates to a transpiration leak protection element in accordance with the preamble of patent claim 1.

Systems for the absorption of sweat in the area of the armpits have been around for many years. In particular, there are many inventions with pads that can be attached to the body or to garments. The disadvantage of these solutions is often that they slip during movement and have to be attached with great effort in such a way that they cannot slip. Another disadvantage of such pads is that they usually have rather small surfaces for practical reasons, so that the absorbing performance is limited, decreases relatively quickly, and that the pad often has to be replaced.

In DE 10 2004 031 561, an undershirt with an integrated armpit moisture protection is presented, so that there is no longer a risk of slipping. Additionally, the presented sewn-in reinforcements can be designed with large surfaces so that the absorbing performance is increased. The disadvantage of such designs is that the undershirt needs to be changed in case of very strong sweating, because the absorbing performance is not sufficient.

The known solutions are mostly limited to the armpit area where strong sweating takes place. The presented solutions are also not suitable for other body parts with strong sweating such as chest or back.

The main object of the present invention is to provide an improved transpiration leak protection element of the type mentioned in the beginning in a way that the advantages of the known pads and undershirts with sewn-in reinforcements remain unchanged, but that the transpiration leak protection element is able to provide the desired absorbing performance even in the case of very strong sweating.

This is achieved by providing a transpiration leak protection element with the features of the patent claim 1. Further features and design examples emerge from the dependent claims, and their advantages are explained in the following description.

Shown in the drawing:

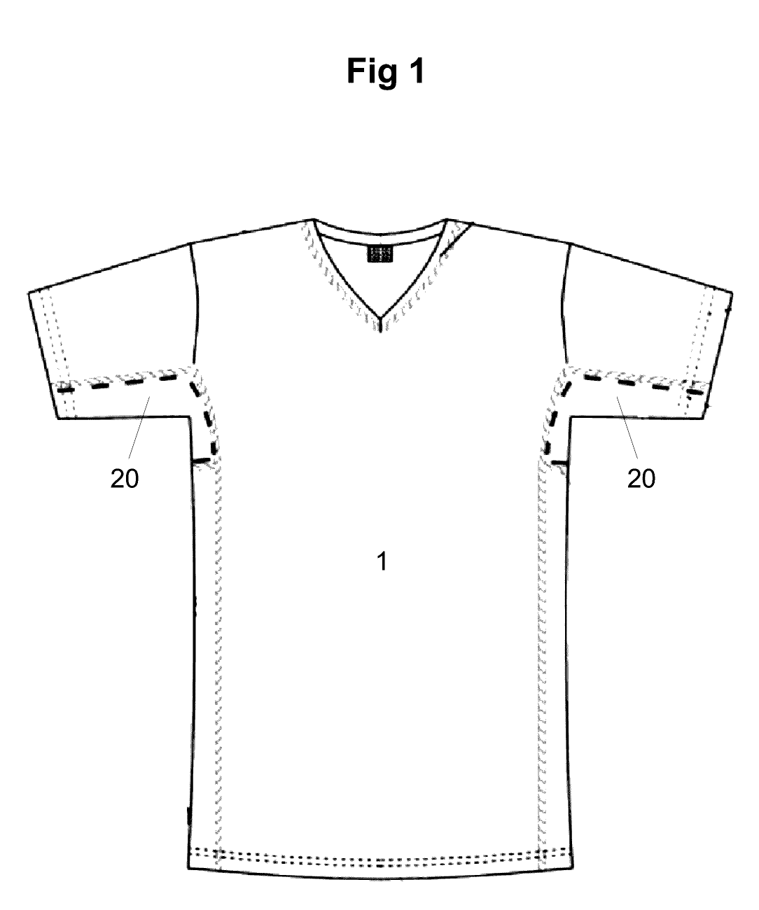

FIG. 1 Shirt

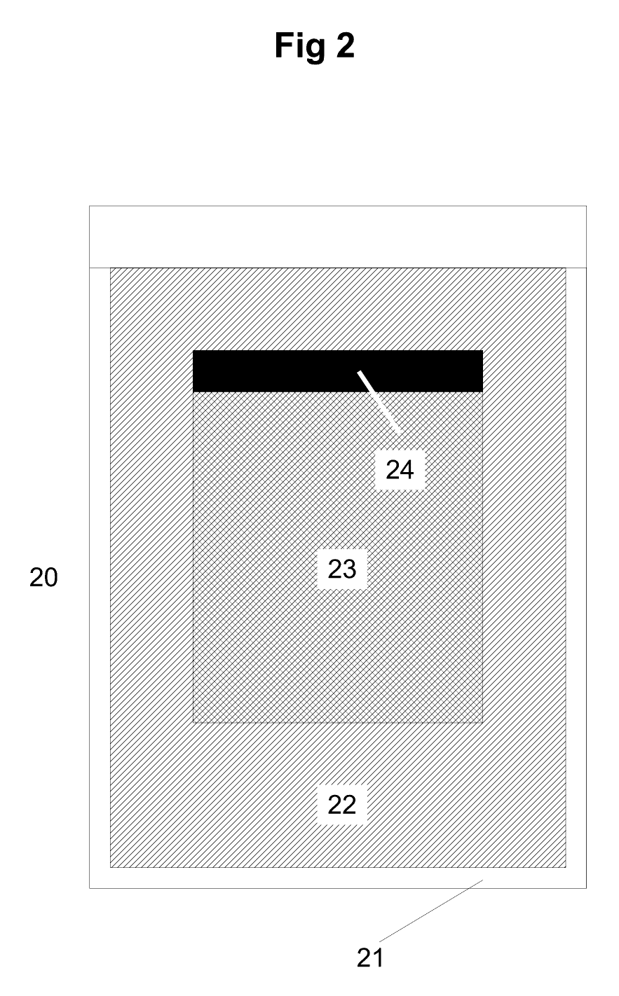

FIG. 2 Transpiration leak protection element

The figures represent possible embodiments which are explained in the following description.

In FIG. 1, a garment 1 is shown which has a transpiration leak protection element 20 in the area of the armpits on both sides. The example of a garment shown here is an undershirt, but the transpiration leak protection element may also be attached to other outer garments such as a shirt, blouse, or sweater.

The design of the transpiration leak protection element 20 is shown schematically in FIG. 2. The individual dimensions and whether the base area is rectangular as shown here or has a different shape like trapezoid or parallelogram or a round shape, depend on which garment the transpiration leak protection element 20 is attached to. The shape and extension of the transpiration leak protection element also depends on the body parts for which it shall be used. For example, it may be larger for a transpiration leak protection element in the chest or back area.

The transpiration leak protection element 20 is attached to the inside of the garment in an appropriate way. One possibility would be to sew it on the garment, but it also may be glued on.

The bottom layer of the transpiration leak protection element 20 is the membrane layer 21. It is totally impermeable to wetness and has a sufficient extension so that all areas where sweating takes place are covered, for example the armpits. The membrane layer 21 consists for example of a material composed of more than 80% polyamide as well as a mix of polyurethanes (PUE and PUR). Any other waterproof materials may also be used.

Due to the water repellent properties the material of the membrane layer 21 is usually not pleasant on the skin so that a cover layer 22 is attached above. This cover layer can consist of the same material as the garment itself, especially if it is an undershirt made of 100% cotton. A combination of cotton with up to 10% spandex is also very well-suited. This layer is very pleasant on the skin and additionally fulfills an absorbent function.

Depending on the extension and thickness of the cover layer 22, the absorbency may vary significantly. However, in the case of strong sweating it can always be expected that, over a longer period, the cover layer 22 is saturated and cannot absorb any more liquid. In order to prevent sweat flowing from the cover layer 22 to the garment, the extension of the cover layer 22 is smaller than the extension of the membrane layer 21, so that liquid cannot reach the garment for example via the seam of the cover layer 22. Nevertheless, in order to achieve the maximum absorbing performance as well as the best comfort for the garment wearer, a distance between the edge of the cover layer 22 and the edge of the membrane layer 21 as small as possible is chosen, as shown in FIG. 2. This ensures that due to the sealing effect of the membrane layer, it would also prevent the occurrence of sweat stains on the garment at the edge of the cover layer 22.

The cover layer 22 is attached to the membrane layer 21 in an appropriate way. This can be achieved, for example, by gluing or sewing, whereby in the case of sewing attention should be paid to using a thread that is also water-repellent, in order to prevent sweat flowing to the garment via the seam which lead through the membrane layer 21. Such a thread also needs to be flexible to a certain extent so that the different extension of the membrane layer 21 and the cover layer 22 does not cause any swelling up.

As the cover layer 22 is not absorbent enough over a longer period in the case of strong sweating and there is not always the opportunity or desire to change the garment as soon as the saturation with sweat is reached, a pocket is attached to the cover layer 22 which may be filled with any desired highly absorbent material. This ensures that sweat is initially absorbed by this highly absorbent material before the absorbent function of the cover layer 22 has to be used.

As soon as the highly absorbent material is saturated with sweat, it can be replaced with relatively little effort, ideally without having to take off the garment 1. For this purpose, the pocket 23 has an opening, through which the highly absorbent material can be replaced.

In order to enable an easy replacement of the highly absorbent material, the opening for a garment with short sleeves is directed towards the outside so that the replacement is possible through the arm opening with the second hand. However, the opening may also be attached to other sides of the pocket or even be in the middle of the pocket formed as a slit.

In order to prevent the highly absorbent material from falling out during movement or even simply due to gravity, the opening may have a fastener which can easily be opened and closed again. Any types of fasteners are possible, for example a zipper or a hook-and-loop fastener.

Specially designed underarm sweating pads or widely available paper tissues can be used as highly absorbent material. On the one hand, the possibility of using any highly absorbent material allows great flexibility in case the normally used material is not available, and on the other, it offers the possibility to use the least expensive material.

According to the type of highly absorbent material used, the pocket 23 is designed large enough for the underarm sweating pads or paper tissues to fit in without slipping, but can also be inserted and taken back out without problems. The extension of the pocket 23 is at most the same size as that of the cover layer 22 to guarantee the functioning of the membrane layer 21 which prevents the occurrence of sweat stains on the garment.

The material of the pocket 23 is chosen in such a way that on the one hand it is pleasant on the wearer's skin and on the other, it can transport the sweat to the highly absorbent material as well as possible. Instead of cotton, the use of mesh fabric made from polyester is very well-suited as it is very permeable to wetness and attracts moisture.

1. Transpiration leak protection element with a membrane layer which is impermeable to wetness, a pocket which is permeable to wetness and a cover layer positioned in between, characterized in that
the cover layer is attached to the membrane layer and has a smaller extension as the membrane layer,
the pocket is attached to the cover layer and has a smaller or same size extension as the cover layer, and the pocket is filled with a highly absorbent material.

2. Transpiration leak protection element according to claim 1, characterized in that
the highly absorbent material is a pad, a compress, a cushion or a folded tissue.

3. Transpiration leak protection element according to claim 1 or 2, characterized in that
the pocket has at least partly an opening on at least one side through which the highly absorbent material is exchanged in the pocket.

4. transpiration leak protection element according to claim 3, characterized in that
the opening has a fastener which is closed and reopened.

5. transpiration leak protection element according to claim 4, characterized in that
the fastener is a hook-and-loop fastener or a zipper.
